(19) United States
(12) Patent Application Publication
(10) Pub. No.: US 2004/0077687 A1
(43) Pub. Date: Apr. 22, 2004

(54) Thiazolmines and their use as tgf-beta inhibitors

(76) Inventor: Gellibert Jeanne Francoise, Les Ulis (FR)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/470,882

(22) PCT Filed: Jan. 31, 2002
(86) PCT No.: PCT/EP02/00993

(51) Int. Cl. A61K 31/444; C07D 417/14

(52) U.S. Cl. 514/333; 546/256

(57) ABSTRACT

Therapeutically active thiazole derivatives of formula (I) wherein R¹ and R² are as defined in the specification, processes for the preparation thereof, the use thereof in therapy, particularly in the treatment or prophylaxis of disorders characterised by overexpression of transforming growth factor β (TGF-β), and pharmaceutical compositions for use in such therapy.

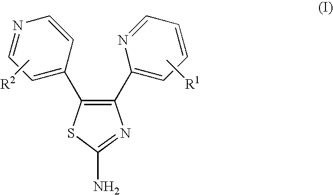

The present invention relates to novel thiazole derivatives, processes for the preparation thereof, the use thereof in therapy, particularly in the treatment or prophylaxis of disorders characterised by overexpression of transforming growth factor β (TGF-β), and pharmaceutical compositions for use in such therapy.

TGF-β is a multi-functional cytokine which belongs to the TGF-β superfamily which includes activins/inhibins, bone morphogenetic proteins (BMPs) and TGF-βs. Three isoforms of TGF-β (TGF-β1, TGF-β2, and TGF-β3) have been identified in mammals, each of which is encoded by a distinct gene on different chromosomes (D. A. Lawrence, Eur. Cytokine. Netw., 1996, 7(3), 363). TGF-β initiates an intracellular signalling pathway which ultimately leads to the expression of genes that regulate cell cycle, control proliferative responses, or relate to extracellular matrix proteins that mediate cell adhesion, migration and intercellular communication. TGF-β has pleitropic effects including modulation of cell growth and differentiation, extracellular matrix formation, hematopoiesis, and immunomodulation (Roberts and Spoon, Handbook of Experimental Pharmacology, 1990, 95, 419-458).

A variety of cell surface proteins and receptors are known to transduce the signals initiated by the binding of the active TGF-β ligand to its receptors. Initiation of the TGF-β signalling pathway results from the binding of the TGF-β ligand to the extracelullar domain of the type II membrane receptor (Massague, Ann. Rev. Biochem., 1998, 67, 753.). The bound type II receptor then recruits type I (Alk5) receptor into a multimeric membrane complex, whereupon active type II receptor kinase phoshorylates and activates type I receptor kinase. The function of the type I receptor kinase is to phosphorylate a receptor-associated co-transcription factor, Smad-2 or Smad-3; thereby releasing it into the cytoplasm where it binds to Smad-4. The PAI-1 gene is activated by TGF-β as a consequence of the above-mentioned cellular pathway.

One approach to the treatment and/or prophylaxis of disorders characterised by the overexpression of TGF-β is inhibition of the TGF-β signal transduction. For example inhibition of the TGF-β type II receptor by overexpression of a dominant negative TGF-β type II receptor has previously been shown to prevent liver fibrosis and dysfunction in rat models (Proc. Natl. Acad. Sci, 1999, 96(5), 2345), and also to prevent progression of established liver fibrosis (Hepatology, 2000, 32, 247).

Pathological overexpression of TGF-β is known to be associated with a number of undesirable effects, leading ultimately to the development of serious pathogenic conditions (G. C. Blobe et al., N. Engl. J. Med., 2000, 1350). In particular, pathological overexpression of TGF-β3 may cause excessive accumulation of extracellular matrix (ECM), inhibition of cell proliferation and immunosupression. Excessive accumulation of ECM is known to lead to fibrotic diseases such as tumor fibrosis, radiation-induced fibrosis, fibrosis of the liver, kidney, lung, bowel, heart, pancreas, peritoneum or other organs. Fibrosis can lead to pathologic conditions such as cirrhosis, idiopathic pulmonary fibrosis, glomerulosclerosis and hypertrophic scars.

A number of other disease states are known to be associated with variations in the expression of genes which are controlled by TGF-β including cancer development, abnormal bone function and inflammatory disorders.

The development of compounds capable of inhibiting the TGF-β intracellular pathway is seen as a desirable way to effect prophylaxis and/or treatment of the above-mentioned conditions. Compounds capable of inhibiting the TGF-β intracellular pathway and/or the expression of TGF-β may be used in the treatment of disorders the symptoms of which often lead to the development of fibrotic conditions. For example, compounds of the present invention may be useful in treating the fibrosis associated with various liver-related conditions such as hepatitis B virus (HBV), hepatitis C virus (HCV), alcohol-induced hepatitis, haemochromatosis and primary biliary cirrhosis, or alternatively may be useful in treating fibrosis associated with various kidney-related conditions such as chronic renal disease, acute renal disease and kidney disease.

The compounds of the present invention are thiazole derivatives. Other thiazole compounds have previously been described for use in alternative medicinal applications. PCT Patent Application WO 96/03392 (Searle & Co) discloses a series of substituted thiazole compounds for the treatment of inflammation and inflammation-related disorders. WO 93/15071 (SmithKline Beecham Intercredit N.V.) describes a series of thiazolyl-pyridine derivatives which may be used as gastric acid secretion inhibitors. This type of compound may be useful in the treatment of gastrointestinal disorders such as gastric and duodenal ulcers, aspiration pneumonitis and Zollinger-Ellison Syndrome. U.S. Pat. No. 5,232,921 (Biziere et al.) discloses 2-alkylaminothiazoles having an affinity for muscarinic cholinergic receptors. None of the aforementioned patent applications describe the thiazole compounds of the present invention.

PCT Patent Application WO 00/12947 (Scios Inc.) describes the use of a series of quinazoline derivatives for treating various disorders associated with enhanced activity of kinase p38-α and/or TGF-β. The compounds described therein have been shown to inhibit the activities of both proteins and are therefore particularly useful for the treatment of conditions in which an enhanced activity towards both p38-α and TGF-β is required.

It has now been discovered that certain substituted thiazole compounds, as described below, are useful in the treatment or prophylaxis of disorders characterised by the overexpression of TGF-β. In particular, compounds of the present invention are TGF-β inhibitors which act at the TGF-β type I (Alk5) receptor level.

According to one aspect of the present invention, we provide compounds of formula (I),

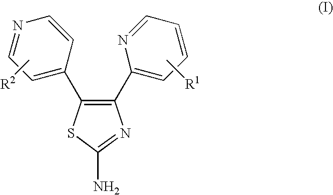

wherein,

R¹ is selected from H, halo (such as fluoro, chloro, bromo), —CN, —CF₃, C₁₋₄ alkyl or C₁₋₄ alkoxy;

R² is selected from phenyl, furanyl or thiophenyl, each of which may be further substituted by one or more substituents, which may be the same or different, selected from halo (such as fluoro, chloro, bromo), —CN, —CF₃, —OCF₃, C₁₋₄ alkyl or C₁₋₄ alkoxy, and

and salts and solvates thereof (hereinafter “compounds of the invention”).

As used herein the term “alkyl” as a group or part of a group refers to a straight or branched chain saturated aliphatic hydrocarbon radical containing the specified number(s) of carbon atoms. Such alkyl groups in particular include methyl, ethyl, n-propyl, iso-propyl, n-butyl, sec-butyl, tert-butyl, pentyl and hexyl.

The term “alkoxy” as a group or part of a group refers to an alkyl ether radical, wherein the term “alkyl” is defined above. Such alkoxy groups in particular include methoxy, ethoxy, n-propoxy, iso-propoxy, n-butoxy, isobutoxy, sec-butoxy and tert-butoxy.

The present invention also covers the physiologically acceptable salts of the compounds of formula (I). Suitable physiologically acceptable salts of the compounds of formula (I) include acid salts, for example sodium, potassium, calcium, magnesium and tetraalkylammonium and the like, or mono- or di-basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like.

The present invention also relates to solvates of the compounds of Formula (I), for example hydrates.

Preferably, R¹ is positioned at the C(3) or C(6) position of the pyridine ring and is selected from H, halo (such as fluoro, chloro, bromo), —CN, —CF₃, C₁₋₄ alkyl or C₁₋₄ alkoxy. More preferably R¹ is H.

Preferably, R² is located at the C(2) position of the pyridinyl ring and represents phenyl or thiophenyl each of which may be further substituted by one or more substituents, which may be the same or different, selected from halo (such as fluoro, chloro, bromo), —CN, —CF₃, C₁₋₄ alkyl, C₁₋₄ alkoxy or —OCF₃. More preferably R² is located at the C(2) position of the pyridinyl ring and is phenyl which may be further substituted by one or more substituents, which may be the same or different, selected from halo (such as fluoro, chloro, bromo), —CN, —CF₃, C₁₋₄ alkyl, C₁₋₄ alkoxy or —OCF₃. Most preferably R² is located at the C(2) position of the pyridinyl ring and is phenyl which may be further substituted at the para-position by a substituent selected from halo (such as fluoro, chloro, bromo), —CN, —CF₃, C₁₋₄ alkyl, C₁₋₄ alkoxy or —OCF₃.

It will be appreciated that the present invention is intended to include compounds having any combination of the preferred groups as listed hereinbefore.

Compounds of formula (I) which are of special interest as agents useful in the treatment or prophylaxis of disorders characterised by the overexpression of TGF-β are,

5-[2-(4-Chlorophenyl)pyridin-4-yl]-4-pyridin-2-yl-1,3-thiazol-2-amine;

5-[2-(4-Methoxyphenyl)pyridin-4-yl]-4-pyridin-2-yl-1,3-thiazol-2-amine;

5-[2-(4-Fluorophenyl)pyridin-4-yl]-4-pyridin-2-yl-1,3-thiazol-2-amine;

5-[2-(4-Ethylphenyl)pyridin-4-yl]-4-pyridin-2-yl-1,3-thiazol-2-amine;

5-[2-(4-Ethoxyphenyl)pyridin-4-yl]-4-pyridin-2-yl-1,3-thiazol-2-amine;

5-[2-(Thiophen-3-yl)pyridin-4-yl]-4-pyridin-2-yl-1,3-thiazol-2-amine; and

and salts and solvates thereof.

Certain compounds of formula (I) may exist in stereoisomeric forms (e.g. they may contain one or more asymmetric carbon atoms or may exhibit cis-trans isomerism). The individual stereoisomers (enantiomers and diastereoisomers) and mixtures of these are included within the scope of the present invention. Likewise, it is understood that compounds of formula (I) may exist in tautomeric forms other than that shown in the formula and these are also included within the scope of the present invention.

Salts of the compounds of the present invention may comprise acid addition salts derived from a nitrogen in the compound of formula (I). The therapeutic activity resides in the moiety derived from the compound of the invention as defined herein and the identity of the other component is of less importance although for therapeutic and prophylactic purposes it is, preferably, pharmaceutically acceptable to the patient. Examples of pharmaceutically acceptable acid addition salts include those derived from mineral acids, such as hydrochloric, hydrobromic, phosphoric, metaphosphoric, nitric and sulphuric acids, and organic acids, such as tartaric, acetic, trifluoroacetic, citric, malic, lactic, fumaric, benzoic, glycolic, gluconic, succinic and methanesulphonic and arylsulphonic, for example β-toluenesulphonic, acids.

Compounds of formula (I) and salts and solvates thereof may be prepared by the methodology described hereinafter, constituting a further aspect of this invention. In yet a further aspect of the present invention there is provided a process for the preparation of intermediate compounds of formula (B).

Compounds of formula (I) may conveniently be prepared according to the general methodology in Scheme I below:

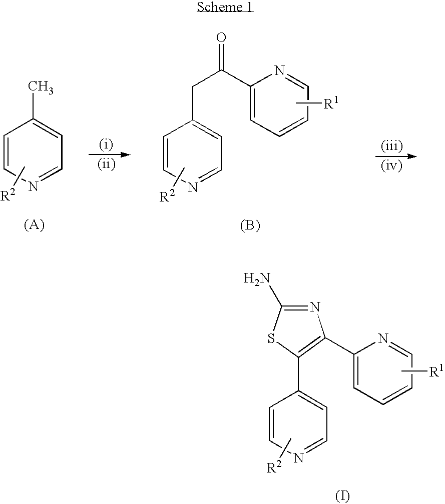

Reagents and conditions (preferred): (i) KHMDS, THF, −50° C.; (ii) R¹(C₅H₃N)CO₂Et, THF, −50° C.; (iii) polymer-supported pyridinium perbromide, THF, r.t.; (iv) thiourea, EtOH, reflux.

### List of Abbreviations

| | |
|---|---|
| EtOH | Ethanol |
| KHMDS | Potassium bis(trimethylsilyl)amide |
| THF | Tetrahydrofuran |

A general process according to the invention for preparing a compound of formula (I) comprises:

(i) Addition of a suitable base, such as potassium bis(trimethylsilyl)amide or sodium bis(trimethylsilyl)amide to a substituted pyridine of formula (A), preferably in the temperature range 0 to −75° C., more preferably in the temperature range −30 to −60° C., most preferably at −50° C., in the presence of a suitable solvent such as THF;

(ii) Addition of a suitable monosubstituted pyridyl ester, R¹(C₅H₅N)CO₂Et (wherein R¹ is hereinbefore defined) to the reaction mixture, preferably in the temperature range 0 to −75° C., more preferably in the temperature range −30 to −60° C., most preferably at −50° C., in the presence of a suitable solvent such as THF;

(iii) Halogenation of the resulting ketone (B) with a suitable halogenating agent, preferably a brominating reagent such as Br₂ or polymer-supported pyridinium perbromide, preferably in the temperature range 0-75° C., more preferably in the temperature range 20 to 60° C., most preferably at room temperature, in the presence of a suitable solvent such as THF; and

(iv) Addition of thiourea in a suitable solvent such as ethanol and heating the mixture under reflux.

Compounds of formula (A) may be prepared by processes analogous to those known in the art (e.g. Osuch et al., J. Org. Chem., 1957, 22, 939). Examples of such preparative procedures are provided in the specific examples hereinafter.

Monosubstituted pyridyl esters, R¹(C₅H₃N)CO₂Et (where R¹ is hereinbefore defined) as described in step (ii) above may be prepared by processes analogous to those known in the art. For example, where R¹=C(6)—OMe, Finger et al., J. Org. Chem., 1962, 27, 3965; where R¹═C(3)—OMe, Dejardin et al., Bull. Chem. Soc. Fr., 1979, 289; where R¹═C(5)-Br, Chambers and Marfat, Synth. Commun., 1997, 27(3), 515; and where R¹═C(4)-CN, Heinisch and Lotsch, Heterocycles, 1987, 26(3), 731.

The compounds of the present invention have been found to inhibit phosphorylation of the Smad-2 or Smad-3 proteins by inhibition of the TGF-β type I (Alk5) receptor.

Furthermore, the compounds of the invention have been tested in the assays described herein and have been found to be of potential therapeutic benefit in the treatment and prophylaxis of disorders characterised by the overexpression of TGF-β.

Thus there is provided a compound of formula (I) or a physiologically acceptable salt or solvate thereof for use as a medicament in human or veterinary medicine, particularly in the treatment or prophylaxis of disorders characterised by the overexpression of TGF-β.

It will be appreciated that references herein to treatment extend to prophylaxis as well as the treatment of established conditions. It will further be appreciated that references herein to treatment or prophylaxis of disorders characterised by the overexpression of TGF-β, shall include the treatment or prophylaxis of TGF-β associated disease such as fibrosis, especially liver and kidney fibrosis, cancer development, abnormal bone function and inflammatory disorders, and scarring.

Other pathological conditions which may be treated in accordance with the invention have been discussed in the introduction hereinbefore. The compounds of the present invention are particularly suited to the treatment of fibrosis and related conditions.

Compounds of the present invention may be administered in combination with other therapeutic agents, for example antiviral agents for liver diseases or in combination with ACE inhibitors or Angiotensin II receptor antagonists for kidney diseases.

According to another aspect of the invention, there is provided the use of a compound of formula (I) or a physiologically acceptable salt or solvate thereof for the manufacture of a medicament for the treatment and/or prophylaxis of disorders characterised by the overexpression of TGF-β, particularly fibrosis.

In a further aspect there is provided a method for the treatment of a human or animal subject with a disorder characterised by the overexpression of TGF-β, particularly fibrosis, which method comprises administering to said human or animal subject an effective amount of a compound of formula (I) or a physiologically acceptable salt or solvate thereof.

Compounds of the invention may be formulated for administration in any convenient way, and the invention therefore also includes within its scope pharmaceutical compositions for use in therapy, comprising a compound of formula (I) or a physiologically acceptable salt or solvate thereof in admixture with one or more physiologically acceptable diluents or carriers.

There is also provided according to the invention a process for preparation of such a pharmaceutical composition which comprises mixing the ingredients.

Compounds of the invention may, for example, be formulated for oral, buccal, parenteral, topical or rectal administration.

Tablets and capsules for oral administration may contain conventional excipients such as binding agents, for example syrup, acacia, gelatin, sorbitol, tragacanth, mucilage of starch or polyvinyl pyrrolidone; fillers, for example, lactose, microcrystalline cellulose, sugar, maize-starch, calcium phosphate or sorbitol; lubricants, for example, magnesium stearate, stearic acid, talc, polyethylene glycol or silica; disintegrants, for example, potato starch, croscarmellose sodium or sodium starch glycollate; or wetting agents such as sodium lauryl sulphate. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxymethyl cellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example almond oil, fractionated coconut oil, oily esters, propylene glycol or ethyl alcohol; or preservatives, for example, methyl or propyl p-hydroxybenzoates or sorbic acid. The preparations may also contain buffer salts, flavouring, colouring and/or sweetening agents (e.g. mannitol) as appropriate.

For buccal administration the compositions may take the form of tablets or lozenges formulated in conventional manner.

The compounds may also be formulated as suppositories, e.g. containing conventional suppository bases such as cocoa butter or other glycerides.

Compounds of the invention may also be formulated for parenteral administration by bolus injection or continuous infusion and may be presented in unit dose form, for instance as ampoules, vials, small volume infusions or pre-filled syringes, or in multi-dose containers with an added preservative. The compositions may take such forms as solutions, suspensions, or emulsions in aqueous or non-aqueous vehicles, and may contain formulatory agents such as anti-oxidants, buffers, antimicrobial agents and/or toxicity adjusting agents. Alternatively, the active ingredient may be in powder form for constitution with a suitable vehicle, e.g. sterile, pyrogen-free water, before use. The dry solid presentation may be prepared by filling a sterile powder aseptically into individual sterile containers or by filling a sterile solution aseptically into each container and freeze-drying.

By topical administration as used herein, we include administration by insufflation and inhalation. Examples of various types of preparation for topical administration include ointments, creams, lotions, powders, pessaries, sprays, aerosols, capsules or cartridges for use in an inhaler or insufflator or drops (e.g. eye or nose drops).

Ointments and creams may, for example, be formulated with an aqueous or oily base with the addition of suitable thickening and/or gelling agents and/or solvents. Such bases may thus, for example, include water and/or an oil such as liquid paraffin or a vegetable oil such as arachis oil or castor oil or a solvent such as a polyethylene glycol. Thickening agents which may be used include soft paraffin, aluminium stearate, cetostearyl alcohol, polyethylene glycols, microcrystalline wax and beeswax.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents or suspending agents.

Spray compositions may be formulated, for example, as aqueous solutions or suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluorethane, carbon dioxide or other suitable gas.

Capsules and cartridges for use in an inhaler or insufflator, of for example gelatin, may be formulated containing a powder mix of a compound of the invention and a suitable powder base such as lactose or starch.

Compounds of the invention may conveniently be administered in amounts of, for example, 0.01 to 100 mg/kg body weight, suitably 0.05 to 25 mg/kg body weight orally, one or more times a day. The precise dose will of course depend on the age and condition of the patient, the particular route of administration chosen, and is entirely within the discretion of the administering physician.

The following non-limiting Examples illustrate the present invention.

Intermediates

Compounds of Formula (A)

Intermediate A1: 2-(4-Chlorophenyl)-4-methyl-pyridine

2-Bromo-4-methylpyridine (ALDRICH, 2.29 g, 13.4 mmol) was dissolved in toluene (30 ml) and lithium chloride (3 eq, 1.64 g) and tetrakis(triphenylphosphine)palladium (0) (5 mol %, 734 mg) added under N₂ and degassed. The mixture was cooled in an ice bath and aqueous sodium carbonate (2M, 2 eq) added slowly and stirred for 10 min. A solution of 4-chlorophenylboronic acid (Lancaster, 2.5 g, 1.2 eq) in ethanol (20 ml) added slowly and stirred, returned to room temp then heated to reflux for 4 h. The mixture was allowed to cool, the phases separated and the aqueous extracted with further toluene (2×50 ml). The combined organic phases were extracted with 1N HCl (2×50 ml). These combined acid extracts were washed with toluene (50 ml) and then basified to pH14 with 1N NaOH with cooling.

The product was then extracted into CH₂Cl₂ (3×50 ml) dried and filtered to give, on concentration, the title compound as a yellow solid (2.6 g).

m.p 63° C.

TLC SiO₂ CH₂Cl₂/EtOAc 95/5 Rf 0.64

GC-MS: 203

Intermediate A2: 2-(4-Methoxyphenyl)-4-methyl-pyridine

A procedure similar to that for Intermediate A1, with substitution of 4-methoxyphenylboronic acid (Lancaster, 15 g) for 4-chlorophenylboronic gave the title compound as an orange oil (14.2 g).

TLC SiO₂ CH₂Cl₂/MeOH 98/2 Rf 0.85

MS(API): 200 (MH+)

Intermediate A3: 2-(4-Fluorophenyl)-4-methyl-pyridine

A procedure similar to that for Intermediate A1, with substitution of 4-fluorophenylboronic acid (Lancaster, 11 g) for 4-chlorophenylboronic acid gave the title compound as a yellow oil (6.5 g).

TLC SiO₂ CH₂Cl₂ Rf 0.20

GC-MS: 187

Intermediate A4: 2-(4-Ethylphenyl)-4-methyl-pyridine

A procedure similar to that for Intermediate A1, with substitution of 4-ethylphenylboronic acid (Lancaster, 4 g) for 4-chlorophenylboronic acid gave the title compound as an oil (3.9 g).

TLC SiO₂ CH₂Cl₂/EtOAc 95/5 Rf 0.50

GC-MS: 197

Intermediate A5: 2-(4-Ethoxyphenyl)-4-methyl-pyridine

A procedure similar to that for Intermediate A1, with substitution of 4-ethoxyphenylboronic acid (Aldrich, 5 g) for 4-chlorophenylboronic acid gave the title compound as an oil (4.3 g).

TLC SiO₂ CH₂Cl₂/EtOAc 95/5 Rf 0.44

GC-MS: 213

Compounds of Formula (B)

Intermediate B1: 2-[2-(4-Chloro-phenyl)-pyridin-4-yl]-1-pyridin-2-yl-ethanone

To a solution of Intermediate A1 (2-(4-chlorophenyl)-4-methyl-pyridine, 2.6 g) in dry THF (30 ml) at −50° C. under argon, a solution of potassium bis-(trimethylsilyl)amide 0.5M in toluene (20 ml, 1.1 eq) was added dropwise. The solution was stirred at this temperature for 1 h, then a solution of ethyl picolinate (2.26 g) in dry THF (20 ml) was added and the reaction mixture was allowed to warm to room temperature overnight then heated to 50° C. for 2 h. The solvent was evaporated under reduced pressure and the solid precipitated with hexane. The brown solid was then taken up in saturated NH₄Cl solution and the aqueous phase was extracted with ethyl acetate. The organic layer was dried over sodium sulfate and concentrated to give the title compound as a dark red oil (2.6 g).

TLC SiO₂ CH₂Cl₂/MeOH 98/2 Rf 0.42

MS(API): 309(MH+)

Intermediate B2: 2-[2-(4-Methoxy-phenyl)-pyridin-4-yl]-1-pyridin-2-yl-ethanone

A procedure similar to that for Intermediate B1, with substitution of Intermediate A2 (5.0 g) for Intermediate A1, gave the title compound as a pale yellow solid (2.0 g).

mp 86° C.

TLC SiO₂ cyclohexane/EtOAc 70/30 Rf 0.37

MS(API): 305(MH+)

Intermediate B3: 2-[2-(4-Fluoro-phenyl)-pyridin-4-yl]-1-pyridin-2-yl-ethanone

A procedure similar to that for Intermediate B1, with substitution of Intermediate A3 (6.5 g) for Intermediate A1, gave the title compound as a dark yellow oil (3.1 g).

TLC SiO₂ CH₂Cl₂/MeOH 95/5 Rf 0.30

MS(API): 293(MH+)

Intermediate B4: 2-[2-(4-Ethylphenyl)-pyridin-4-yl]-1-pyridin-2-yl-ethanone

A procedure similar to that for Intermediate B1, with substitution of Intermediate A4 (3.9 g) for Intermediate A1, gave the title compound as a yellow oil (1.2 g).

TLC SiO₂ CH₂Cl₂/EtOAc 95/5 Rf 0.32

GC-MS: 302

Intermediate B5: 2-[2-(4-Ethoxyphenyl)-pyridin-4-yl]-1-pyridin-2-yl-ethanone

A procedure similar to that for Intermediate B1, with substitution of Intermediate A5 (4.3 g) for Intermediate A1, gave the title compound as an oil (1.2 g).

TLC SiO₂ CH₂Cl₂/MeOH 98/2 Rf 0.24

GC-MS: 318

Intermediate B6: 2-[2-(Thiophen-3-yl-pyridin-4-yl]-1-pyridin-2-yl-ethanone

A procedure similar to that for Intermediate B1, with substitution of 3-methyl-2-thiophen-3-yl-pyridine (HFR, 2 g) for Intermediate A1, gave the title compound as an oil (0.7 g).

TLC SiO₂ Cyclohexane/EtOAc 60/40 Rf 0.38

MS(API): 281(MH+)

### EXAMPLES

### Example 1

### 5-[2-(4-Chlorophenyl)pyridin-4-yl]-4-pyridin-2-yl-1,3-thiazol-2-amine

To a solution of Intermediate B1 (0.62 g) in THF (20 ml) was added polymer-supported pyridinium perbromide (Aldrich, 1 eq) and the suspension shaken for 50 min. The resin was removed by filtration, with the filtrate being added directly to thiourea (1 eq) and the resin washed many times with ethanol. The filtrate was heated at reflux for overnight, allowed to cool and concentrated. The residue was basified with aqueous NaOH, extracted into CH₂Cl₂.and this phase washed with water. The organic phase was dried, concentrated and triturated with ether to give a solid which was recrystallised from EtOH to give the title compound as off-white crystals (100 mg).

m.p 256-257° C.

TLC SiO₂ CH₂Cl₂/MeOH 98/2 Rf 0.69

MS(API): 365(MH+)

### Example 2

### 5-[2-(4-Methoxyphenyl)pyridin-4-yl]-4-pyridin-2-yl-1,3-thiazol-2-amine

A procedure similar to that for Example 1 using Intermediate B2 (1.0 g) gave, after recrystallisation from EtOAc, the title compound as an off-white solid (850 mg).

mp 222° C.

TLC SiO₂ toluene/isopropylamine 90/10 Rf 0.31

MS(API): 361(MH+)

### Example 3

### 5-[2-(4-Fluorophenyl)pyridin-4-yl]-4-pyridin-2-yl-1,3-thiazol-2-amine

A procedure similar to that for Example 1 using Intermediate B3 (0.9 g) gave, after recrystallisation from methanol, the title compound as white crystals (150 mg).

m.p 250° C.

TLC SiO₂ CH₂Cl₂/MeOH 90/10 Rf 0.15

MS(API): 349(MH+)

### Example 4

### 5-[2-(4-Ethylphenyl)pyridin-4-yl]-4-pyridin-2-yl-1,3-thiazol-2-amine

A procedure similar to that for Example 1 using Intermediate B4 (366 mg) gave, after recrystallisation from ethanol, the title compound as white crystals (100 mg).

m.p 238° C.

TLC SiO₂ CH₂Cl₂/MeOH 90/10 Rf 0.34

MS(API): 359(MH+)

### Example 5

### 5-[2-(4-Ethoxyphenyl)pyridin-4-yl]-4-pyridin-2-yl-1,3-thiazol-2-amine

A procedure similar to that for Example 1 using Intermediate B5 (387 mg) gave, after recrystallisation from ethanol, the title compound as white crystals (150 mg).

m.p 246° C.

TLC SiO₂ CH₂Cl₂/MeOH 90/10 Rf 0.4

MS(API): 375(MH+)

### Example 6

### 5-[2-(Thiophen-3-yl)pyridin-4-yl]-4-pyridin-2-yl-1,3-thiazol-2-amine

A procedure similar to that for Example 1 using Intermediate B6 (0.65 g) gave, after recrystallisation from ethanol, the title compound as a white powder (200 mg).

m.p 244° C.

TLC SiO₂ CH₂Cl₂/MeOH 98/2 Rf 0.5

MS(API): 337(MH+)

Biological Data

The compounds of Examples 1-6 were tested in vitro, using the biological assays described below. All of the compounds had an IC₅₀ value of 5 μM or below in Assay 1, and an IC₅₀ value of 1 μM or below in Assay 2.

Assays

Assay 1

The potential for compounds of the invention to inhibit TGF-β signalling may be demonstrated, for example, using the following in vitro assay.

The assay was performed in HepG2 cells stably transfected with the PAI-1 promoter (known to be a strong TGF-β responsive promoter) linked to a luciferase (firefly) reporter gene. The compounds were selected on their ability to inhibit luciferase activity in cells exposed to TGF-β. In addition cells were transfected with a second luciferase (Renilla) gene which was not driven by a TGF-β responsive promoter and was used as a toxicity control.

(96 well-)microplates are seeded, using a multidrop apparatus, with the stably transfected cell line at a concentration of 35000 cells per well in 200 μl of serum-containing medium. These plates are placed in a cell incubator.

18 to 24 hours later (Day 2), cell-incubation procedure is launched. Cells are incubated with TGF-β and a candidate compound (TGF-β inhibitor) at concentrations in the range 50 nM to 10 μM (final concentration of DMSO 1%). The final concentration of TGF-β (rhTGFβ1) used in the test is 1 ng/mL. Cells are incubated with a candidate compound 15-30 mins prior to the addition of TGF-β. The final volume of the test reaction is 150 μl. Each well contains only one candidate compound and its effect on the PAI-1 promoter is monitored.

Columns 11 and 12 are employed as controls. Column 11 contains 8 wells in which the cells are incubated in the presence of TGF-β, without a candidate compound. Column 11 is used to determine the ‘reference TGF-β induced firefly luciferase value’ against which values measured in the test wells (to quantify inhibitory activity) may be compared. In wells A12 to D12, cells are grown in medium without TGF-β. The firefly luciferase values obtained from these positions are representive of the ‘basal firefly luciferase activity’. In wells E12 to H12, cells are incubated in the presence of TGF-β and 500 μM CPO (Cyclopentenone, Sigma), a cell toxic compound. The toxicity is revealed by decreased firefly and renilla luciferase activities (around 50% of those obtained in column 11).

12 to 18 hours later (day 3), the luciferase quantification procedure is launched. The following reactions are performed using reagents obtained from a Dual Luciferase Assay Kit (Promega). Cells are washed and lysed with the addition of 10 μl of passive lysis buffer (Promega). Following agitation (15 to 30 mins), luciferase activities of the plates are read in a dual-injector luminometer (BMG lumistar). For this purpose, 50 μl of luciferase assay reagent and 50 μl of ‘Stop & Glo’ buffer are injected sequentially to quantify the activities of both luciferases. Data obtained from the measurements are processed and analysed using suitable software. The mean Luciferase activity value obtained in wells A11 to H11 (Column 11, TGF-βonly) is considered to represent 100% and values obtained in wells A12 to D12 (cells in medium alone) give a basal level (0%). For each of the compounds tested, a concentration response curve is constructed from which an IC₅₀ value can be determined graphically.

Assay 2

The potential for compounds of the invention to inhibit the kinase Alk5 receptor may be demonstrated, for example, using the following in vitro assay.

The kinase domain of Alk5 was cloned and expressed in a baculovirus/Sf9 cells system. The protein (amino acids 162 to 503) was 6-His tagged in C-terminus. After purification by affinity chromatography using a Ni²⁺ column, the autophosphorylation was tested. The enzyme was incubated in a medium containing: Tris 50 mM pH 7.4; NaCl 100 mM; MgCl₂ 5 mM; MnCl₂ 5 mM; DTT 10 mM. The enzyme was preincubated with the compounds (0.1% DMSO final in the test) 10 minutes at 37° C. The reaction was initialised by the addition of 3 μM ATP (0.5 μCi gamma-³³P-ATP). After 15 minutes at 37° C. the reaction was stopped by addition of SDS-PAGE sample buffer (50 mM Tris-HCl, pH 6.9, 2.5% glycerol, 1% SDS, 5% beta-mercaptoethanol). The samples were boiled for 5 minutes at 95° C. and run on a 12% SDS-PAGE. The dried gels were exposed to a phosphor screen over-night. Alk5 autophosphorylation was quantified using a STORM (Molecular Dynamics).

1. A compound of formula (I), 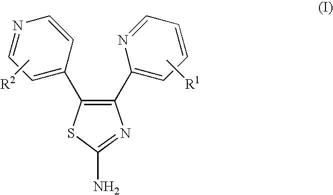
wherein,
R¹ is selected from H, halo, —CN, —CF₃, C₁₋₄ alkyl or C₁₋₄ alkoxy;
R² is selected from phenyl, furanyl or thiophenyl, each of which may be further substituted by one or more substituents, which may be the same or different, selected from halo, —CN, —CF₃, —OCF₃, C₁₋₄ alkyl or C₁₋₄ alkoxy,
and salts and solvates thereof.

2. A compound of formula (I) as claimed in claim 1, wherein R¹ is positioned at the C(3) or C(6) position of the pyridine ring and is selected from H, halo, —CN, —CF₃, C₁₋₄ alkyl or C₁₋₄ alkoxy.

3. A compound of formula (I) as claimed in claim 2, wherein R¹ is H.

4. A compound of formula (I) as claimed in any one of claims 1 to 3, wherein R² is located at the C(2) position of the pyridyl ring and represents phenyl or thiophenyl each of which may be further substituted by one or more substituents, which may be the same or different, selected from halo (such as fluoro, chloro, bromo), —CN, —CF₃, C₁₋₄ alkyl, C₁₋₄ alkoxy or —OCF₃.

5. A compound of formula (I) as claimed in any one of the preceding claims wherein R² is located at the C(2) position of the pyridyl ring and represents phenyl which may be further substituted at the para-position by a substituent selected from halo (such as fluoro, chloro, bromo), —CN, —CF₃, C₁₋₄ alkyl, C₁₋₄ alkoxy or —OCF₃.

6. A compound of formula (I) as claimed in claim 1 selected from:
5-[2-(4-Chlorophenyl)pyridin-4-yl]-4-pyridin-2-yl-1,3-thiazol-2-amine;
5-[2-(4-Methoxyphenyl)pyridin-4-yl]-4-pyridin-2-yl-1,3-thiazol-2-amine;
5-[2-(4-Fluorophenyl)pyridin-4-yl]-4-pyridin-2-yl-1,3-thiazol-2-amine;
5-[2-(4-Ethylphenyl)pyridin-4-yl]-4-pyridin-2-yl-1,3-thiazol-2-amine;
5-[2-(4-Ethoxyphenyl)pyridin-4-yl]-4-pyridin-2-yl-1,3-thiazol-2-amine;
5-[2-(2-Thiophen-3-yl)pyridin-4-yl]-4-pyridin-2-yl-1,3-thiazol-2-amine; and
and salts and solvates thereof.

7. A pharmaceutical composition comprising at least one compound of formula (I) as claimed in any one of claims 1 to 6, together with a pharmaceutically acceptable diluent or carrier.

8. A compound of formula (I) as claimed in any one of claims 1 to 6 for use as a medicament.

9. The use of a compound of formula (I) as claimed in any one of claims 1 to 6, for the manufacture of a medicament for the treatment and/or prophylaxis of disorders characterised by the overexpression of TGF-β.

10. A method for the treatment of a human or animal subject with a disorder characterised by the overexpression of TGF-β, which method comprises administering to said human or animal subject an effective amount of a compound of formula (I) as claimed in any one of claims 1 to 6 or a physiologically acceptable salt or solvate thereof.

11. A process for the preparation of a compound of formula (I), 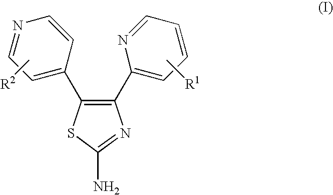
wherein,
R¹ is selected from H, halo, —CN, —CF₃, C₁₋₄ alkyl or C₁₋₄ alkoxy;
R² is selected from phenyl, furanyl or thiophenyl, each of which may be further substituted by one or more substituents, which may be the same or different, selected from halo, —CN, —CF₃, —OCF₃, C₁₋₄ alkyl or C₁₋₄ alkoxy,
and salts and solvates thereof,
which process comprises:
a) addition of a suitable halogenating agent to a compound of formula (B), 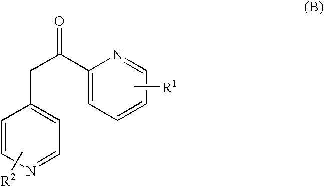
where R¹ and R² are as defined above; and
(b) subsequent addition of thiourea to the resulting reaction mixture.
